(19) United States
(12) Patent Application Publication
(10) Pub. No.: US 2015/0147771 A1
(43) Pub. Date: May 28, 2015

(54) MICROPERFUSION TISSUE INTERROGATOR

(71) Applicant: Cleveland State University, Cleveland, OH (US)

(72) Inventors: Andrew H. Resnick, Shaker Heights, OH (US); Ulrich Hopfer, Cleveland Heights, OH (US); Christian A. Zorman, Euclid, OH (US)

(21) Appl. No.: 14/547,744

(22) Filed: Nov. 19, 2014

Related U.S. Application Data
(60) Provisional application No. 61/907,833, filed on Nov. 22, 2013.

(51) Int. Cl. G01N 33/50

(52) U.S. Cl. CPC G01N 33/5091
USPC 435/29; 435/287.1

(57) ABSTRACT

A cell layer measurement device includes a first zone, a second zone, and a porous membrane between the first and second zones. A tissue sample includes the cell layer on the porous membrane. A fluid is on at least a first side of the tissue. A fluid inflow line and fluid outflow line are on at least a first side of the tissue. At least one electrode measures a property of the fluid. A property of the cell layer is determined based on the measured property of the fluid.

### BACKGROUND

The present invention relates to fabricating micro-electromechanical systems (BioMEMS) devices. It finds particular application in conjunction with BioMEMS devices for epithelia cultured in vitro and will be described with particular reference thereto. It will be appreciated, however, that the invention is also amenable to other applications.

Conventional ways to maintain differentiated epithelia in vitro do not include providing micro-devices. Such micro-devices allow investigators to interrogate epithelial cells in a controlled manner. It is desirable to maintain differentiated epithelia in vitro and assess their functions and thereby understand the principles that govern integration of molecular events to cellular functions. Currently, measuring properties of a tissue sample involves direct measurements of the tissue sample. In other words, the properties are measured by a probe that directly contacts and penetrates into the tissue sample. Different levels of penetration by the probe into the tissue sample may easily result in inconsistent readings.

The present invention provides a new and improved apparatus and method which addresses the above-referenced problems.

### SUMMARY

In one aspect of the present invention, it is contemplated to that a cell layer measurement device includes a first zone, a second zone, and a porous membrane between the first and second zones. A tissue sample includes the cell layer on the porous membrane. A fluid is on at least a first side of the tissue. A fluid inflow line and fluid outflow line are on at least a first side of the tissue. At least one electrode measures a property of the fluid. A property of the cell layer is determined based on the measured property of the fluid.

### BRIEF DESCRIPTION OF THE DRAWINGS

In the accompanying drawings which are incorporated in and constitute a part of the specification, embodiments of the invention are illustrated, which, together with a general description of the invention given above, and the detailed description given below, serve to exemplify the embodiments of this invention.

FIG. 1 illustrates a schematic representation of a cell layer measurement device in accordance with one embodiment of an apparatus illustrating principles of the present invention;

FIG. 2 illustrates a schematic representation of a cross-sectional view of the cell layer measurement device of FIG. 1 in accordance with one embodiment of an apparatus illustrating principles of the present invention;

FIG. 3 illustrates a schematic representation of a plurality of cell layer measurement devices in accordance with one embodiment of an apparatus illustrating principles of the present invention;

FIG. 4 illustrates a schematic representation of a cell layer measurement device in accordance with another embodiment of an apparatus illustrating principles of the present invention; and

FIG. 5 is an exemplary methodology of measuring a property of a cell layer in accordance with one embodiment illustrating principles of the present invention.

### DETAILED DESCRIPTION OF ILLUSTRATED EMBODIMENT

FIG. 1 illustrates a simplified component diagram of an exemplary cell layer measurement device 10 in accordance with one embodiment of the present invention. FIG. 2 illustrates a cross-sectional view of the exemplary cell layer measurement device 10 of FIG. 1. In one embodiment, the device 10 is a micro-device and is used for measuring epithelial cells. A micro-device provides for measuring properties of relatively smaller sample sizes. For example, sample sizes may include only one cell, a few thousand cells, or up to thousands of cells (e.g., up to about 5,000 cells), as opposed to at least hundreds of thousands of cells (e.g., about 300,000 cells) that are required in typical conventional measurement devices. Such smaller sample sizes provide for lower cost measurements, particularly for more expensive samples.

With reference to FIGS. 1 and 2, the cell layer measurement device 10 includes a first zone 12 and a second zone 14. In one embodiment, the first zone 12 is a lower zone, and the second zone 14 is an upper zone. A porous membrane 16₁ is between the first zone 12 and the second zone 14. In one embodiment, the porous membrane 16₁ is a polycrystalline silicon (polysilicon) film into which a highly dense array of vertical channels may be fabricated. It is contemplated that the porous membrane 16₁ is about 5 microns thick. However, other embodiments, in which the porous membrane 16₁ has different thicknesses, are also contemplated.

A sample 22₁ is on the porous membrane 16₁ with a first cell attachment layer 20₁. For example, the sample 22₁ is adhered to the porous membrane 16₁ and faces the first zone 12. Different ways of adhering the sample 22₁ to the porous membrane 16₁ are contemplated. For example, the sample 22₁ may be adhered to the porous membrane 16₁ by extracellular adhesion molecules as the first cell attachment (e.g., adhesion) layer 20₁. In one embodiment, the sample 22₁ is a two-dimensional tissue sample. As discussed above, the first cell attachment layer 20₁ may include epithelial cells, each of which has an area between about 10 μm ² and about 100 μm². The tissue sample may be a single layer of cells. However, alternate embodiments including a plurality of cell layers are also contemplated. For example, an additional cell attachment layer 20₂, which may be the same as or different from the first cell attachment layer 20₁, may be included on a side of the porous membrane 16₁ facing the first zone 12. The first and second cell attachment layers 20₁, 20₂, respectively, are collectively referred to by reference numeral 20.

A fluid 24 is on at least a first side 26 of the tissue sample 22₁. The first side 26 of the tissue sample 22₁ is a face of the tissue sample 22₁ toward the first zone 12 (e.g., lower zone). A second side 30 of the tissue sample 22₁ is a face of the tissue sample 22₁ toward the second zone 14 (e.g., upper zone). For example, in one embodiment, the fluid 24₁ is at least in the first zone 12 (e.g., lower zone). The fluid 24₁ in the first zone 12 (e.g., lower zone) contacts the first side 26 of the tissue sample 22₁. Other embodiments, in which a fluid 24₂, which may be the same as or different from the fluid 24₁, is also in the second zone 14 (e.g., upper zone), are also contemplated. Fluid 24₂ in the second zone 14 contacts the second side 30 of the tissue sample 22₁. If the fluid 24_{1,2} is in both the first and second zones 12, 14, respectively, the fluid 24_{1,2} contacts both the first and second sides 26, 30, respectively, of the tissue sample 22₁. In other words, the fluid 24 surrounds the tissue sample 22₁. Fluid 24_{1,2} in both the first and second zones 12, 14, respectively, is collectively referenced as 24. Fluids 24₁ and 24₂ can be identical or different.

The fluid 24 is passed into at least one of the first and second zones 12, 14, respectively, of the device 10 from a first reservoir 32 via a first fluid line 34. Therefore, the first fluid line 34 is referred to as a fluid inflow line. In one embodiment, the first fluid line 34 fluidly communicates with the first zone 12 of the device 10. As the fluid 24 is passed from the first reservoir 32 to the first zone 12, the fluid 24 begins to fill the first zone 12. In one embodiment, once the first zone 12 is filled, the fluid 24 begins to fill the second zone 14, provided absence of a confluent cell layer. A second fluid line 36 fluid communicates the fluid 24 from the device 10 to, for example, at least one of the first reservoir 32 and a second reservoir 40. Therefore, the second fluid line 36 is referred to as a fluid outflow line. For example, the second fluid line 36 receives the fluid 24 from at least one of the first and second zones 12, 14, respectively. The level and volume of the fluid 24 in the first and second zones 12, 14, respectively, of the device 10 may be controlled by where at least one of the first and second fluid lines 34, 36, respectively, is attached to the device 10. For example, if the second fluid line 36 is attached to the second zone 14 of the device 10, the fluid 24 will fill the first zone 12 and at least partially fill the second zone 14 of the device 10 (e.g., up to the level where the second fluid line 36 attached to the device 10). It is contemplated that the fluid 24 is circulated through the device 10 by entering the device 10 via the first fluid line 34 and exiting the device 10 via the second fluid line 36. If the first and second reservoirs 32, 40 are fluidly connected to each other, thereby creating, in essence, a single reservoir, the fluid 24 is re-circulated through the device 10. Alternatively, fluid transmitted to the second reservoir 40 is collected for analysis and/or disposal.

In one embodiment, at least one of the fluid 24₁ and the fluid 24₂ is/are a liquid. However, other embodiments including fluids that are not liquid are also contemplated. For example, it is also contemplated that the fluid 24₂ is moist air.

At least one electrode 42 measures a property of the fluid 24. For example, the electrode 42 measures at least one of a pH of the fluid 24, an electrical property of the fluid 24, and electrical potential of the fluid 24. In another embodiment, an additional electrode serves as a ground electrode and completes the circuit. Other embodiments may include more electrodes.

In the illustrated embodiment, two (2) of the electrodes 42₁, 42₂ are positioned on first and second sides 44, 46, respectively, of the device 10. As seen in FIG. 2, the first electrode 42₁ includes two (2) electrode pads 50_{1,1}, 50_{1,2}, and the second electrode 42₂ includes two (2) electrode pads 50_{2,1}, 50_{2,2}. As illustrated, each of the electrodes 42 includes one pad 50_{1,1}, 50_{2,1} in the first zone 12 (e.g., lower zone) and the other pad 50_{1,2}, 50_{2,2} in the second zone 14 (e.g., upper zone). For example, the first pad 50_{1,1} of the first electrode 42₁ is in the first zone 12 (e.g., lower zone) and the second pad 50_{1,2} of the first electrode 42₁ is in the second zone 14 (e.g., upper zone). Similarly, the first pad 50_{2,1} of the second electrode 42₂ is in the first zone 12 (e.g., lower zone) and the second pad 50_{2,2} of the second electrode 42₂ is in the second zone 14 (e.g., upper zone).

With reference again to FIGS. 1 and 2, the first and second electrodes 42₁, 42₂ are at a substantially fixed position relative to the porous membrane 16₁. For example, the first electrode 42₁ is about 0.1 millimeter from a first edge 52 of the porous membrane 16₁, and the second electrode 42 is about 0.1 millimeter from a second edge 54 of the porous membrane 16₁. Providing a substantially fixed position of the first and second electrodes 42ₖ, 42₂ relative to the respective edges 52, 54 of the porous membrane 16₁ results in more consistent readings and results.

The first and second zones 12, 14, respectively, the porous membrane 16₁, the tissue sample 22ₖ, the fluid 24, and the electrodes 42ₖ, 42₂ in the first and second zones 12, 14, respectively, are included in a first chamber 56₁ of the device 10. In other words, the first chamber 56₁ includes a volume defined by the first and second zones 12, 14, respectively, and volumes of the porous membrane 16₁, the tissue sample 22ₖ, the fluid 24, and the electrodes 42ₖ, 42₂ between or in the first and second zones 12, 14, respectively. In one embodiment, the first chamber 56₁ is about 1.5 mm in diameter and about 200 μm in height and has a volume of ˜0.5 mm³.

In one embodiment, the first chamber 56₁ is an integrated (e.g., monolithic) structure. Such integrated (monolithic) structures are relatively easier to manufacture, sterilize, and/or dispose of after use.

Cell activity on the cell attachment layers 20 is not diffusion limited. More specially, mass transport by the cells on the cell attachment layers 20 is faster than diffusion. In other words, the cell activity on the cell layers 20 is flow limited (e.g., reaction limited or kinetically limited). In one example, a diffusion of solute within the first and second zones 12, 14, respectively, has a Peclet number Pe>1.

With reference to FIG. 3, it is also contemplated to include a plurality of the devices 10. The first device 10₁ includes a first zones 12, a second zone (not illustrated in FIG. 3), a porous membrane 16₁, and a tissue sample 22₁, as discussed above. Similar to the first device 10₁, the second device 10₂ includes a third zone 66, a fourth zone (not illustrated in FIG. 3) corresponding to the second zone of the first device 10₁, a porous membrane 16₂, and a tissue sample 22₂. The porous membrane 16₁ and the porous membrane 16₂ are collectively referenced as 16.

In one embodiment, the first device 10₁ receives the fluid 24 from the first reservoir 32 via the first (e.g., inflow) fluid line 34. The fluid 24 is delivered from the first device 10₁ to the second reservoir 40 via the second (e.g., outflow) fluid line 36. The fluid 24 from the second reservoir 40 is delivered to the second device 10₂ via a third (e.g., inflow) fluid line 60. The fluid 24 is delivered from the second device 10₂ to a third reservoir 62 via a fourth (e.g., outflow) fluid line 64. Although only two of the devices 10₁, 10₂ are illustrated in FIG. 3, it is to be understood that any number of the devices 10 may be included.

As discussed above, the first zone 12, the second zone (not illustrated in FIG. 3), the first porous membrane 16₁, the first tissue sample 22ₖ, the fluid 24, and the electrodes 42ₖ, 42₂ are included in the first chamber 56₁ of the first device 10₁. Similarly, the third zone 66, the fourth zone (not illustrated in FIG. 3), the second porous membrane 16₂, the second tissue sample 22₂, the fluid 24, and the electrodes 42₃, 42₄ are included in the second chamber 56₂ of the second device 10₂. In other words, the second chamber 56₂ includes a volume defined by the third zone 66 and the fourth zone (not illustrated in FIG. 3), and volumes of the porous membrane 16₂, the tissue sample 22₂, the fluid 24, and the electrodes 42₃, 42₄ between or in the third zone 66 and the fourth zone (not illustrated in FIG. 3). Like the first chamber 56₂ discussed above, in one embodiment, the second chamber 56₂ is also about 1.5 mm in diameter and about 200 μm in height and has a volume of ˜0.5 mm³.

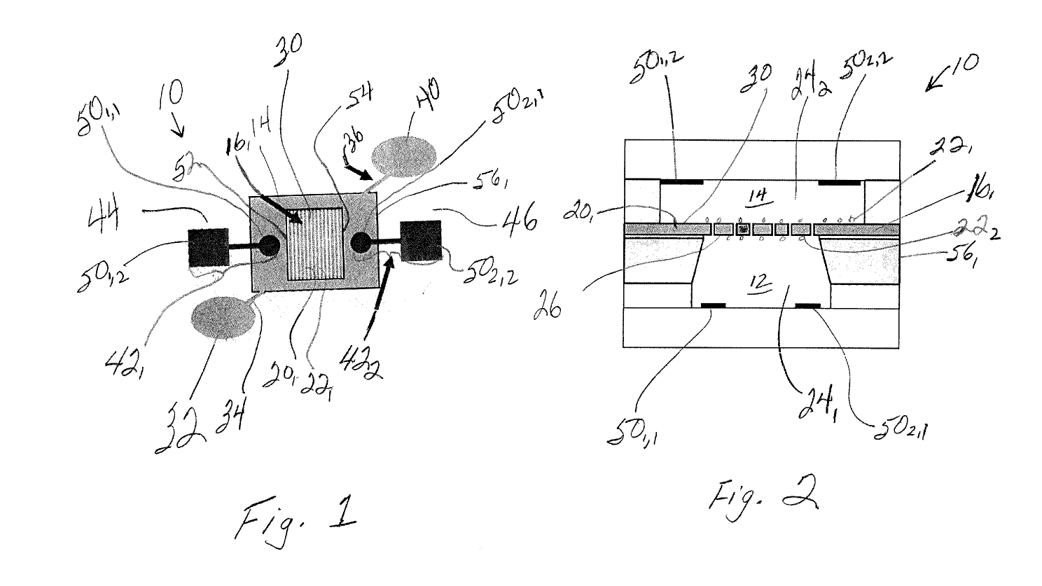

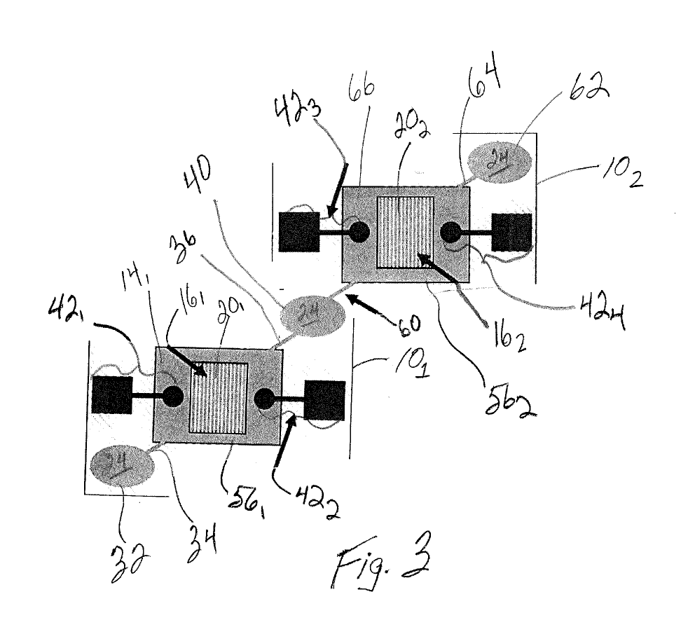

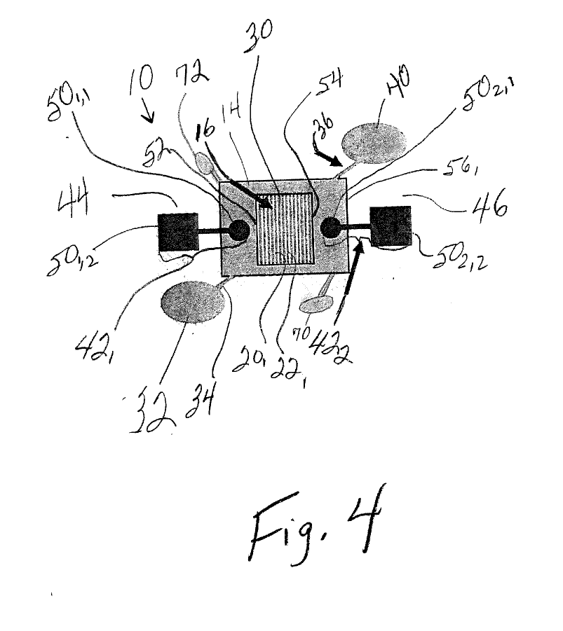

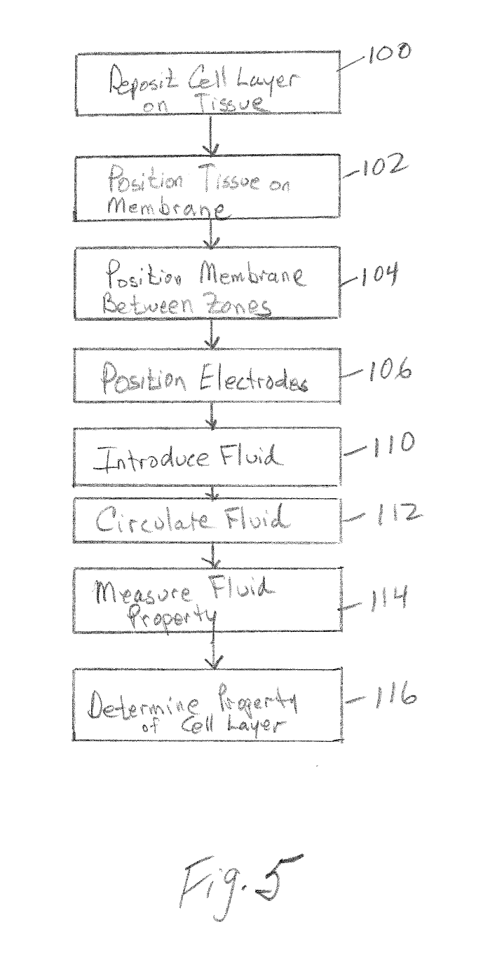

With reference to FIG. 4, in another embodiment, the fluid 24 passes from the first reservoir 32 to a first delivery reservoir 70. Also, the fluid 24 passes from a first source reservoir 72 to the second reservoir 40. In this embodiment, it is contemplated that the fluid 24₁ passes from the first reservoir 32 to the first delivery reservoir 70 via the first zone 12 without, for example passing through the second zone 14. It is also contemplated that the fluid 24₂ passes from the first source reservoir 72 to the second reservoir 40 via the second zone 14 without, for example passing through the first zone 12.

With reference to FIG. 5, an exemplary methodology of the system shown in FIGS. 1 and 2 for measuring a property of a cell layer on a tissue sample 22₁ and/or 22₂ (22) is illustrated. As illustrated, the blocks represent functions, actions and/or events performed therein. It will be appreciated that electronic and software systems involve dynamic and flexible processes such that the illustrated blocks and described sequences can be performed in different sequences. It will also be appreciated by one of ordinary skill in the art that elements embodied as software may be implemented using various programming approaches such as machine language, procedural, object-oriented or artificial intelligence techniques. It will further be appreciated that, if desired and appropriate, some or all of the software can be embodied as part of a device's operating system.

In a step 100, the cell layer is deposited on the tissue sample 22. In a step 102, the tissue sample 22 is positioned on the porous membrane 16. For example, the tissue sample 22 is adhered to the porous membrane 16. The porous membrane 16 is positioned between the first zone 12 and the second zone 14 in the chamber 56 of the device 10, in a step 104. The first and second electrodes 42ₖ, 42₂ are positioned in the device 10 in a step 106. For example, as discussed above, the first and second electrodes 42₁, 42₂ are positioned at fixed positions relative to the porous membrane 16.

The fluid 24 is introduced into at least one of the first zone 12 and the second zone 14 in a step 110. In one embodiment, as discussed above, the 24 is introduced to surround the tissue sample 22. In a step 112, the fluid 24 is circulated through the device 10. If multiple devices 10₁, 10₂ are used, the fluid 24 is circulated from the first chamber 56₁ of the first device 10₁ to the second chamber 56₂ of the second device 10₂.

A property of the fluid 24 is measured in a step 114. The property of the fluid 24 measured in the step 114 includes, for example, at least one of: pH of the fluid 24, osmolarity of the fluid 24, an electrical property of the fluid 24, etc.

A property of the cell layer on the tissue sample 22 is determined in a step 116. For example, the property of the cell layer is determined in the step 116 based on the property of the fluid measured in the step 114.

It is to be understood that although the steps described above generally apply to a single device 10, similar steps apply if multiple devices are used 10.

The attached Appendix includes additional details and embodiments of the present invention.

While the present invention has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the invention, in its broader aspects, is not limited to the specific details, the representative apparatus, and illustrative examples shown and described. Accordingly, departures may be made from such details without departing from the spirit or scope of the applicant's general inventive concept.

What is claimed is:

1. A cell layer measurement device, comprising:
a first zone;
a second zone;
a porous membrane between the first and second zones;
a tissue sample including the cell layer on the porous membrane;
a fluid on at least a first side of the tissue;
a fluid inflow line and a fluid outflow line on at least a first side of the tissue; and
at least one electrode measuring a property of the fluid, a property of the cell layer being determined based on the measured property of the fluid.

2. The cell layer measurement device as set forth in claim 1, wherein:
a diffusion of solute near the cell layer has a Peclet number Pe>1.

3. The cell layer measurement device as set forth in claim 2, wherein:
a first chamber including the first and second zones, the porous membrane, the tissue sample, the fluid, and the at least one electrode in the first and second zones has a volume of ˜0.5 mm³.

4. The cell layer measurement device as set forth in claim 1, wherein:
the first zone is a lower zone; and
the second zone is an upper zone.

5. The cell layer measurement device as set forth in claim 4, wherein:
the fluid is the first zone.

6. The cell layer measurement device as set forth in claim 5, wherein:
the fluid is in both the first and second zones; and
the fluid surrounds the tissue sample.

7. The cell layer measurement device as set forth in claim 1, wherein the property of the fluid measured by the at least one electrode includes at least one of a pH of the fluid, an osmolarity of the fluid, and an electrical property of the fluid.

8. The cell layer measurement device as set forth in claim 1, wherein the fluid is a liquid.

9. The cell layer measurement device as set forth in claim 1, wherein:
the tissue sample is adhered to the porous membrane.

10. The cell layer measurement device as set forth in claim 1, wherein:
the at least one electrode is at a fixed position relative to the porous membrane.

11. The cell layer measurement device as set forth in claim 1, wherein:
the first side of the porous membrane is in the first zone; and
the second side of the porous membrane is in the second zone.

12. The cell layer measurement device as set forth in claim 1, wherein the first zone, the second zone, the porous membrane, the tissue sample, the fluid and the at least one electrode are in a first chamber, the cell layer measurement device further including:
a second chamber including:
a third zone;
a fourth zone;
a second porous membrane; and
a second tissue sample.

13. The cell layer measurement device as set forth in claim 12, wherein:
the fluid is in at least one of the first and second zones; and
the fluid is in at least one of the third and fourth zones.

14. The cell layer measurement device as set forth in claim 12, wherein:
the fluid is in at least one of the first and second zones; and
a second fluid is in at least one of the third and fourth zones.

15. A method for measuring a property of a cell layer, the method comprising:
depositing the cell layer on an adhesion layer of a tissue sample;
adhering the adhesion layer to a porous membrane;
introducing a fluid into at least one of the first zone and the second zone;
measuring a property of the fluid; and
determining a property of the cell layer based on the measured property of the fluid.

16. The method for measuring a property of a cell layer as set forth in claim 15, wherein the measuring step includes:
measuring at least one of a pH of the fluid and an electrical property of the fluid.

17. The method for measuring a property of a cell layer as set forth in claim 15, further including:
positioning at least one electrode at a fixed position relative to the porous membrane.

18. The method for measuring a property of a cell layer as set forth in claim 15, further including:
introducing the fluid to surround the tissue sample.

19. The method for measuring a property of a cell layer as set forth in claim 15, further including:
circulating the fluid from a first chamber including the tissue sample to a second chamber including a second tissue sample.

20. The method for measuring a property of a cell layer as set forth in claim 19, wherein:
measuring a property of the fluid includes:
measuring a property of the fluid in the first chamber;
the method further including:
depositing a second cell layer on an adhesion layer of a second tissue sample;
adhering the adhesion layer of the second tissue sample to a porous membrane;
measuring a second property of the fluid in the second chamber; and
determining a second property of the second cell layer based on the second measured property of the fluid.
